(19) United States
(12) Patent Application Publication
(10) Pub. No.: US 2014/0039417 A1
(43) Pub. Date: Feb. 6, 2014

(54) COMPOSITE MATERIAL AND INDUSTRIAL ENDOSCOPE

(71) Applicant: Olympus Corporation, Tokyo (JP)

(72) Inventors: Naoyuki Osako, Tokyo (JP); Takeshi Kida, Tokyo (JP); Kohei Oguni, Tokyo (JP)

(73) Assignee: Olympus Corporation, Tokyo (JP)

(21) Appl. No.: 14/050,864

(22) Filed: Oct. 10, 2013

Related U.S. Application Data
(63) Continuation of application No. PCT/JP2012/066296, filed on Jun. 26, 2012.

(30) Foreign Application Priority Data
Jul. 12, 2011 (JP) 2011-153643

(51) Int. Cl. C08L 75/06; A61B 1/00; C08L 9/02

(52) U.S. Cl. CPC C08L 75/06; C08L 9/02; A61B 1/00135
USPC 604/264; 525/127

(57) ABSTRACT

A composite material includes nitrile group-containing copolymer rubber; polyester-based thermoplastic polyurethane; and polyalkylene oxide containing metal salt, in which the metal salt is alkali metal salt or alkaline earth metal salt, and an amount of the metal salt in the polyalkylene oxide containing metal salt is 0.5 parts by mass to 3.0 parts by mass with respect to 100 parts by mass of the total amount of the nitrile group-containing copolymer rubber and the polyester-based thermoplastic polyurethane.

### BACKGROUND OF THE INVENTION

1. Field of the Invention

The present invention relates to a composite material and an industrial endoscope.

This application claims priority of and the benefit of Japanese Patent Application No. 2011-153643 filed on Jul. 12, 2011, and is a continuous application of international application PCT/JP2012/066296 filed on Jun. 26, 2012, the disclosures thereof are incorporated herein by reference.

2. Description of Related Art

A flexible material having rubber elasticity is used in various fields because molding such as extrusion molding or injection molding can be easily performed with the flexible material.

In particular, superior gasoline resistance is required for a flexible material used for an industrial endoscope applied for observation and inspection on internal damages and corrosions within various machines including nuclear reactors, and parts for automobiles etc. In addition, the flexible material is required to not deteriorate with exposure to ozone (ozone resistance).

As a flexible material having superior gasoline resistance and ozone resistance, a polymer alloy disclosed in Japanese Patent Application Publication No. 2004-91506 which contains a nitrile group-containing copolymer rubber (for example, nitrile rubber (NBR)) and an acrylic resin, and a polymer alloy disclosed in Japanese Patent Application Publication No. 2004-59695 which contains NBR and urethane rubber are proposed.

### SUMMARY OF THE INVENTION

According to a first aspect of the present invention, a composite material includes nitrile group-containing copolymer rubber; polyester-based thermoplastic polyurethane; and polyalkylene oxide containing metal salt, in which the metal salt is alkali metal salt or alkaline earth metal salt, and an amount of the metal salt in the polyalkylene oxide containing metal salt is 0.5 parts by mass to 3.0 parts by mass with respect to 100 parts by mass of a total amount of the nitrile group-containing copolymer rubber and the polyester-based thermoplastic polyurethane.

According to a second aspect of the present invention, in the composite material according to the first aspect, the polyester-based thermoplastic polyurethane may be crosslinked with the polyalkylene oxide containing metal salt.

According to a third aspect of the present invention, an industrial endoscope according to the first aspect may include a member obtained by molding the composite material according to the first aspect.

According to a fourth aspect of the present invention, in the industrial endoscope according to the third aspect, the member may be a tube which covers an outer surface of an insertion portion used to be inserted into a tissue.

### BRIEF DESCRIPTION OF THE DRAWINGS

FIG. 1 is a perspective view of an industrial endoscope according to an embodiment of the present invention.

### DETAILED DESCRIPTION OF THE INVENTION

Hereinafter, an embodiment of the present invention will be described.

### [Composite Material]

A composite material according to the embodiment contains nitrile group-containing copolymer rubber; polyester-based thermoplastic polyurethane; and polyalkylene oxide containing metal salt.

### <Nitrile Group-Containing Copolymer Rubber>

The nitrile group-containing copolymer rubber is rubber containing nitrile groups in the molecules thereof. The nitrile group-containing copolymer rubber has little crosslinking points between polymer chains so the polymer chains are able to move freely. Therefore, the nitrile group-containing copolymer rubber has superior flexibility.

The composite material exhibits superior rubber elasticity by containing the nitrile group-containing copolymer rubber.

The nitrile group-containing copolymer rubber is obtained by copolymerization of an unsaturated nitrile monomer and another monomer which is copolymerizable with the unsaturated nitrile monomer.

Examples of the unsaturated nitrile monomer include acrylonitrile and methacrylonitrile.

The amount of the unsaturated nitrile monomer unit in the nitrile group-containing copolymer rubber is preferably 30 mass % to 50 mass % and more preferably 30 mass % to 40 mass %. When the amount of the unsaturated nitrile monomer unit is higher than or equal to 30 mass %, gasoline resistance is improved. On the other hand, when the amount of the unsaturated nitrile monomer unit is lower than or equal to 50 mass %, low-temperature properties (cold resistance) are improved.

Examples of the another monomer include a conjugated diene monomer, an unconjugated diene monomer, α-olefine, and an aromatic vinyl monomer.

Examples of the conjugated diene monomer include 1,3-butadiene, 2-methyl-1,3-butadiene, and 1,3-pentadiene.

Examples of the unconjugated diene monomer include 1,4-hexanediene, ethylidene norbornene, and dicyclopentadiene.

Examples of α-olefine include ethylene, propylene, 1-butene, and 1-methylpentene.

Examples of the aromatic vinyl monomer include styrene and α-methylstyrene.

As the nitrile group-containing copolymer rubber, a copolymer of acrylonitrile and 1,3-butadiene (nitrile rubber: NBR) is preferable from the viewpoints of obtaining superior flexibility and easily being mixed with the polyester-based thermoplastic polyurethane as described below.

Examples of NBR which can be used include NBR (extremely high-nitrile NBR) having an acrylonitrile content (bound acrylonitrile content, hereinafter referred to as “bound AN content”) of 42% or higher, NBR (high-nitrile NBR) having a bound AN content of 36% to lower than 42%, NBR (medium high-nitrile NBR) having a bound AN content of 31% to lower than 36%, NBR (medium-nitrile NBR) having a bound AN content of 25% to lower than 31%, and NBR (low-nitrile NBR) having a bound AN content of lower than 25%. NBR may be used according to the use of the composite material because properties thereof change depending on the bound AN content. For example, when the composite material is used for an industrial endoscope for investigating the inside of various machines, a nuclear reactor, or the like or for a member such as automobile parts (particularly, peripheral parts of an engine), NBR having a high bound AN content is preferable. In addition, when the composite material is used for a member used in a low-temperature environment, NBR having a low bound AN content is preferable.

The nitrile group-containing copolymer rubber can be obtained with a known method by copolymerization of the unsaturated nitrile monomer and the another monomer.

In addition, as the nitrile group-containing copolymer rubber, a commercially available product can be used. Examples of the commercially available product of NBR include “JSR” series (manufactured by JSR Corporation) and “Nipol” series (manufactured by ZEON Corporation).

The amount of the nitrile group-containing copolymer rubber is preferably 50 mass % to 90 mass % and more preferably 60 mass % to 80 mass % with respect to 100 mass % of the composite material. When the amount of the nitrile group-containing copolymer rubber is higher than or equal to 50 mass %, superior rubber elasticity of the obtained composite material can be maintained well. On the other hand, when the amount of the nitrile group-containing copolymer rubber is lower than or equal to 90 mass %, ratios of the polyester-based thermoplastic polyurethane and the polyalkylene oxide containing metal salt which are described below can be sufficiently secured. Therefore, superior gasoline resistance, ozone resistance, low-temperature properties, and antistatic properties of the obtained composite material can be maintained well.

### <Polyester-Based Thermoplastic Polyurethane>

The polyester-based thermoplastic polyurethane (hereinafter, also referred to as “polyester-based TPU”) has chemically strong urethane bonds (—NHCOO—) in the molecules thereof. Therefore, the polyester-based thermoplastic polyurethane has many intermolecular hydrogen bonds having a high bonding strength and has superior gasoline resistance. In addition, since the polyester TPU does not have a double bond between carbon atoms in the molecules thereof, a molecular structure is not likely to change due to exposure to ozone, and ozone resistance is superior. Furthermore, the polyester-based TPU has excellent low-temperature properties.

The composite material can exhibit superior gasoline resistance, ozone resistance, and low-temperature properties by containing the polyester-based TPU.

For example, when ether-based thermoplastic polyurethane is used instead of the polyester-based TPU, the gasoline resistance of the composite material deteriorates.

The polyester-based TPU can be obtained by reaction of polyester polyol with diisocyanate compound.

For example, the polyester polyol may be obtained by polycondensation of a dicarboxylic acid and a diol. In addition, the polyester polyol may be obtained by ring-opening polymerization of lactones. However, the polyester polyol is not limited to these examples.

Examples of the dicarboxylic acid include phthalic acid, isophthalic acid, terephthalic acid, maleic acid, fumaric acid, adipic acid, and sebacic acid.

Examples of the diol include ethylene glycol, propylene glycol, tetramethylene glycol, 1,4-butanediol, and 1,6-hexanediol.

Examples of the diisocyanate compound include tolylene diisocyanate, hexamethylene diisocyanate, xylene diisocyanate, hydrogenated xylene diisocyanate, hydrogenated dicyclohexylmethane diisocyanate, and isophorone diisocyanate.

In addition, as the polyester-based TPU, a commercially available product can be used.

Examples of the commercially available product include No. 1000 series, No. 2000 series, No. 3000 series, and No. 8000 series of “KURAMIRON U” (manufactured by Kuraray Co., Ltd.).

The amount of the polyester-based TPU is preferably 10 mass % to 50 mass % and more preferably 20 mass % to 40 mass % with respect to 100 mass % of the composite material. When the amount of the polyester-based TPU is higher than or equal to 10 mass %, superior gasoline resistance, ozone resistance, and low-temperature properties of the obtained composite material can be maintained well. On the other hand, when the amount of the polyester-based TPU is lower than or equal to 50 mass %, the ratio of the nitrile group-containing copolymer rubber described above and the polyalkylene oxide containing a metal salt described below can be sufficiently secured. Therefore, superior rubber elasticity and antistatic properties of the obtained composite material can be maintained well.

In addition, the polyester-based TPU is incorporated into the composite material such that the mass ratio of the polyester-based TPU to the above-described nitrile group-containing copolymer rubber (nitrile group-containing copolymer rubber:polyester-based TPU) is preferably 50:50 to 90:10 and more preferably 60:40 to 80:20. When the ratio of the polyester-based TPU is too low, gasoline resistance, ozone resistance, and low-temperature properties of the composite material tend to deteriorate. On the other hand, when the ratio of the polyester-based TPU is too high, the rubber elasticity of the composite material tends to deteriorate.

### <Polyalkylene Oxide Containing Metal Salt>

The polyalkylene oxide containing metal salt is highly electrically conductive because the metal salt functions as an electrolyte. Therefore, the polyalkylene oxide containing metal salt functions as an ion conductive agent and imparts antistatic properties to the composite material. The composite material to which antistatic properties is imparted allows charged static electricity to escape and prevents the generation of static electricity.

Examples of polymer component (polyalkylene oxide component) which is the major component of the polyalkylene oxide containing metal salt include polyethylene oxide (PEO), polypropylene oxide (PPO), and copolymers thereof (PEO-PPO).

The metal salt is alkali metal salt or alkaline earth metal salt.

Examples of the alkali metal salt include lithium perchlorate, lithium trifluoromethanesulfonate, sodium perchlorate, potassium perchlorate, and lithium salt of an organic boron complex.

Examples of the alkaline earth metal salt include magnesium perchlorate and calcium perchlorate.

As the polyalkylene oxide containing metal salt, a commercially available product can be used. Examples of the commercially available product include a “PEL” series (manufactured by Japan Carlit Co., Ltd.).

The composite material contains the polyalkylene oxide containing metal salt such that the amount of the metal salt in the polyalkylene oxide is 0.5 parts by mass to 3.0 parts by mass with respect to 100 parts by mass of the total amount of the nitrile group-containing copolymer rubber and the polyester-based TPU. When the amount of the metal salt is higher than or equal to 0.5 parts by mass, conductivity is exhibited, and thus a composite material having superior antistatic properties can be obtained. On the other hand, when the amount of the metal salt is higher than 3.0 parts by mass, antistatic properties is not further improved. Furthermore, when the amount of the metal salt increases, the ratio of the polyalkylene oxide component in the composite material inevitably increases. Therefore, the obtained composite material is in the oil form, and cannot be molded into a desired shape.

The amount of the metal salt is more preferably 1.0 part by mass to 2.5 parts by mass.

### <Crosslinking Agent>

It is preferable that the composite material according to the embodiment further contain a crosslinking agent. When the crosslinking agent is contained, the polyester-based TPU is crosslinked with the polyalkylene oxide containing metal salt. Therefore, the bleed-out of the polyalkylene oxide containing metal salt can be effectively suppressed. Accordingly, since the metal salt is more uniformly and stably dispersed in the composite material, conductivity is exhibited for a long period of time, and antistatic properties of the composite material can be maintained for a long period of time. In addition, since the bleed-out of the polyalkylene oxide containing metal salt is suppressed, an external appearance of a member formed from the composite material can be satisfactorily maintained.

Examples of the crosslinking agent include a polyol compound, an isocyanate compound, and an isocyanuric acid compound.

Examples of the polyol compound include polycarbonate polyol, polyether polyol, and polyester polyol.

Examples of the isocyanate compound include 4,4′-diphenylmethane diisocyanate, isophorone diisocyanate, xylene diisocyanate, and hexamethylene diisocyanate.

Examples of the isocyanuric acid compound include triallyl isocyanurate, tris-2-hydroxyethyl isocyanurate, tris carboxyethyl isocyanurate, and diallyl monoglycidyl isocyanurate.

The amount of the crosslinking agent is preferably 3 parts by mass to 10 parts by mass with respect to 100 parts by mass of the total amount of the nitrile group-containing copolymer rubber and the polyester-based TPU. When the amount of the crosslinking agent is lower than 3 parts by mass, the polyester-based TPU is not sufficiently crosslinked with the polyalkylene oxide containing a metal salt. On the other hand, when the amount of the crosslinking agent is higher than 10 parts by mass, the moldability of the composite material tends to deteriorate.

### <Preparation Method>

The composite material according to the first embodiment of the present invention can be prepared using various commonly-used methods. For example, the nitrile group-containing copolymer rubber, the polyester-based TPU, and the polyalkylene oxide containing metal salt, and optionally, the crosslinking agent and other components are mixed with each other using a kneading machine such as a two-axis roll, a kneader, or a Banbury mixer, thereby preparing the composite material. In addition, the polyester-based TPU is crosslinked with the polyalkylene oxide containing metal salt in advance using the crosslinking agent to obtain a crosslinked material. Then, this crosslinked material and the nitrile group-containing copolymer rubber, and optionally, other components may be mixed with each other.

The nitrile group-containing copolymer rubber is easily mixed with the polyester-based TPU or with the crosslinked material of the polyester-based TPU and the polyalkylene oxide containing metal salt. Accordingly, the composite material according to the embodiment can be easily prepared as compared to the flexible material disclosed in Japanese Patent Application Publication No. 2004-59695 in which rubbers are mixed with each other.

In addition, since the polyester-based TPU is highly compatible with the polyalkylene oxide component in the polyalkylene oxide containing metal salt, these materials are uniformly mixed. Accordingly, the metal salt in the polyalkylene oxide containing metal salt is likely to be uniformly dispersed in the composite material, and superior antistatic properties can be exhibited.

When the metal salt alone is incorporated into the composite material, it is difficult to uniformly disperse the metal salt in the composite material. Therefore, conductivity is not sufficiently exhibited, and antistatic properties deteriorate.

The above-described composite material according to the embodiment contains the nitrile group-containing copolymer rubber, the polyester-based TPU, and the polyalkylene oxide containing metal salt, and thus can be easily prepared. In addition, the composite material according to the embodiment has rubber elasticity and superior gasoline resistance, ozone resistance, and antistatic properties.

The composite material according to the embodiment can be formed in a desired shape by ordinary molding such as extrusion molding or injection molding.

The composite material according to the embodiment can be used for various applications. The composite material according to the embodiment has rubber elasticity and superior gasoline resistance, ozone resistance, and antistatic properties. Therefore, particularly, the composite material according to the embodiment is suitably used for an industrial endoscope which is used when scratches, corrosion, and the like inside various machines, a nuclear reactor, or the like are observed and inspected. In addition, the composite material according to the embodiment is also suitably used for a member of automobile parts such as a fuel hose or for a material of various members, which require a countermeasure against static electricity, such as a fuel tank cap, a tube, or a hose.

### [Industrial Endoscope]

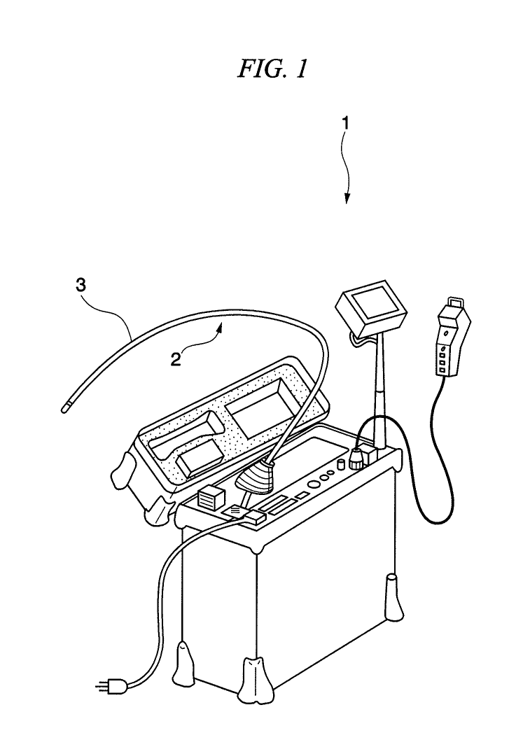

FIG. 1 is a perspective view of an industrial endoscope according to the embodiment of the present invention. The industrial endoscope 1 according to an embodiment of the present invention includes a member obtained by molding the composite material according to the above-described embodiment.

Since this member is required to have rubber elasticity, a tube 3 which covers an outer surface of an insertion portion 2 which is to be inserted into a tissue is preferable as the member.

The industrial endoscope according to the embodiment includes the member obtained by molding the composite material according to the embodiment which has superior gasoline resistance, ozone resistance, and antistatic properties. Therefore, the industrial endoscope according to the embodiment is suitably used as an endoscope for observing and inspecting scratches, corrosion, and the like inside various machines, a nuclear reactor, or the like, specifically, inside a boiler, a turbine, an engine, a chemical plant, or the like.

### EXAMPLES

Hereinafter, examples of the present invention will be described in detail, but the present invention is not limited thereto.

Raw materials used in examples and comparative examples, and evaluation methods are as follows.

### [Raw Materials]

### <Nitrile Group-Containing Copolymer Rubber>

Extremely high-nitrile NBR: Copolymer of acrylonitrile and 1,3-butadiene (“JSR N215SL”, manufactured by JSR Corporation, bound AN content: 49%)
Medium-nitrile NBR: Copolymer of acrylonitrile and 1,3-butadiene (“JSR N241”, manufactured by JSR Corporation, bound AN content: 29%)

### <Thermoplastic Polyurethane>

Polyester-based TPU: Polyester-based thermoplastic polyurethane (“KURAMIRON U8165”, manufactured by Kuraray Co., Ltd.).
Polyether-based TPU: Polyether-based thermoplastic polyurethane (“GUMTHANE AR650”, manufactured by Okada Engineering)

### <Acrylic Resin>

PMMA: Polymethylmethacrylate (“PANLITE L-1250Y” manufactured by Teijin Chemicals Ltd.)

### <Polyalkylene Oxide Containing Metal Salt>

Li salt-containing PAO: Copolymer of polyethylene oxide containing 10% of lithium perchlorate and polypropylene oxide (“PEL-20A” manufactured by Japan Carlit Co., Ltd.)
Mg salt-containing PAO: Solid polyelectrolyte obtained by dissolving magnesium perchlorate in polyethylene oxide such that the amount thereof is approximately 10%.

### <Crosslinking Agent>

Polycarbonate polyol: (“HC-231” manufactured by Nippon Polyurethane Industry Co., Ltd.)
Polyether polyol: (“PANDEX GCB-41” manufactured by DIC Corporation)

### [Evaluation Methods]

### <Evaluation of Antistatic Properties>

Antistatic properties of a test specimen (length 150 mm×width 150 mm×thickness 2 mm or larger) were evaluated as follows.

A test specimen was placed on an insulating plate (length 300 mm×width 300 mm×thickness 10 mm or larger). A surface resistance meter (“ST-3”, manufactured by Simco Japan) was installed on the test specimen to measure the surface resistance value of the test specimen. The lower the surface resistance value, the higher the antistatic properties. Regarding a test specimen of which the surface resistance value could not be measured with the above-described method, by using an insulation resistance tester (“1507”, manufactured by Fluke Corporation) instead of the surface resistance meter, the insulation resistance value of the test specimen was measured, and the measured value was converted into the surface resistance value.

### <Evaluation of Ozone Resistance>

Ozone resistance of a test specimen (length 60 mm×width 10 mm×thickness 2 mm) was evaluated as follows according to JIS-K6259.

The test specimen to which a tensile strain (20% elongation) was given was exposed to air under conditions of an ozone concentration of 50 pphm and a temperature of 40° C. The state of the specimen was observed by visual inspection after 24 hours, 48 hours, 72 hours, and 96 hours. The specimen was classified as follows according to the crack state, and ozone resistance was evaluated.

NC: No cracks were observed

A1, A2, B1, B2, C1, C2: Cracks were observed, but the specimen was not cut off. In addition, the letters represent the number of cracks, in which B is larger than A, and C is larger than B. In addition, the numbers added to the end of the letters represent the size of cracks, in which the larger the number, the larger the size of cracks.

Cut: Large cracks were observed, and the test specimen was cut off

### <Evaluation of Low-Temperature Properties>

The brittle temperature of a test specimen (length 60 mm×width 10 mm×thickness 2 mm) was measured according to JIS-K6301. The lower the brittle temperature, the higher the low-temperature properties.

### <Evaluation of Gasoline Resistance>

Gasoline resistance of a test specimen ((length 60 mm×width 10 mm×thickness 2 mm) was evaluated as follows according to JIS-K6258.

The test specimen was dipped in gasoline JIS No. 1 (25° C.), and after 72 hours, a volume change degree (AV (%)) was obtained. The smaller the volume change degree, the higher the gasoline resistance.

### <Evaluation of Rubber Elasticity>

Rubber elasticity of a test specimen ((length 150 mm×width 25 mm×thickness 6 mm) was evaluated as follows according to JIS-K6260.

O (Good): No cracks, or cracks having a length of less than 1.0 mm were observed

X (Poor): cracks having a length of 1.0 mm or greater were observed

### Example 1

50 parts by mass of extremely high-nitrile NBR as the nitrile group-containing copolymer rubber, 50 parts by mass of polyester-based TPU as the thermoplastic polyurethane, and 25 parts by mass of Li salt-containing PAO (amount of the metal salt: 2.5 parts by mass) as the polyalkylene oxide containing metal salt were mixed with each other using a two-axis kneader to prepare a composite material.

The obtained composite material was molded into a predetermined size using a injection molding machine. As a result, a molded product (test specimen) was prepared. Regarding the obtained test specimen, antistatic properties, ozone resistance, low-temperature properties, gasoline resistance, and rubber elasticity were evaluated. The results are shown in Table 1.

### Examples 2 to 5, Comparative Examples 1 to 7

Composite materials were prepared with the same preparation method as that of Example 1, except that the formulation of the respective components was changed as shown in Table 1. Test specimens were prepared using the obtained composite materials and were evaluated. The results thereof are shown in Table 1.

TABLE 1

| | | | Example | | | | | Comparative Example | | | | | | |
|---|---|---|---|---|---|---|---|---|---|---|---|---|---|---|
| | | | 1 | 2 | 3 | 4 | 5 | 1 | 2 | 3 | 4 | 5 | 6 | 7 |
| Composition | Nitrile | Extremely | 50 | 50 | 50 | 0 | 50 | 100 | 0 | 50 | 50 | 50 | 50 | 50 |
| (Part(s) by | Group- | High-Nitric | | | | | | | | | | | | |
| Mass) of | Containing | NBR | | | | | | | | | | | | |
| Composite | Copolymer | Medium-Nitrile NBR | 0 | 0 | 0 | 50 | 0 | 0 | 0 | 0 | 0 | 0 | 0 | 0 |
| Material | Rubber | | | | | | | | | | | | | |
| | Thermoplastic | Polyester-Based TPU | 50 | 50 | 50 | 50 | 50 | 0 | 100 | 0 | 50 | 50 | 0 | 50 |
| | Polyurethane | Polyether-Based TPU | 0 | 0 | 0 | 0 | 0 | 0 | 0 | 50 | 0 | 0 | 0 | 0 |
| | Acrylic Resin | PMMA | 0 | 0 | 0 | 0 | 0 | 0 | 0 | 0 | 0 | 0 | 50 | 0 |
| | Polyalkylene | Li Salt-Containing PAO | 25 | 25 | 0 | 10 | 5 | 10 | 10 | 10 | 1 | 100 | 25 | 50 |
| | Oxide | Mg Salt-Containing PAO | 0 | 0 | 25 | 0 | 0 | 0 | 0 | 0 | 0 | 0 | 0 | 0 |
| | Containing | | | | | | | | | | | | | |
| | Metal Salt | | | | | | | | | | | | | |
| | Crosslinking | Polycarbonate Polyol | 0 | 3 | 0 | 0 | 0 | 0 | 0 | 0 | 0 | 0 | 0 | 0 |
| | Agent | Polyether Polyol | 0 | 0 | 5 | 0 | 0 | 0 | 0 | 0 | 0 | 0 | 0 | 0 |
| Amount (Part(s) by Mass) of Metal Salt | | | 2.5 | 2.5 | 2.5 | 1.0 | 0.5 | 1.0 | 1.0 | 1.0 | 0.1 | 10.0 | 2.5 | 5.0 |
| Evaluation | Antistatic | Surface Resistance | | | | | | | | | | | | |
| | Properties | Value [10 × Ω] | 6.1 | 7.2 | 8.1 | 7.4 | 8.6 | 7.2 | 8.4 | 8.6 | 15.3 | — | 13.0 | — |
| | | X = | | | | | | | | | | | | |
| | Ozone Resistance | After 24 Hours | NC | NC | NC | NC | NC | A2 | NC | NC | NC | — | B2 | — |
| | | After 48 Hours | NC | NC | NC | NC | NC | A2 | NC | NC | NC | — | Cut | — |
| | | After 72 Hours | NC | NC | NC | NC | NC | B2 | NC | NC | NC | — | Cut | — |
| | | After 96 Hours | NC | NC | NC | NC | NC | B2 | NC | NC | NC | — | Cut | — |
| | Low-Temperature | Brittle Temperature | −22 | −25 | −27 | −31 | −20 | −11 | No | −21 | −20 | — | −25 | — |
| | Properties | [° C.] | | | | | | | Brittling | | | | | |
| | Gasoline | Volume Change | 17 | 13 | 11 | 16 | 14 | 25 | 6 | 25 | 14 | — | 12 | — |
| | Resistance | Degree [%] | | | | | | | | | | | | |

| | | | | | | | | | | | | |
|---|---|---|---|---|---|---|---|---|---|---|---|---|
| Rubber Elasticity | ◯ | ◯ | ◯ | ◯ | ◯ | ◯ | X | ◯ | ◯ | — | ◯ | — |

In Table 1, “Amount of Metal Salt” represents the amount of the metal salt in the polyalkylene oxide containing a metal salt with respect to 100 parts by mass of the total amount of the nitrile group-containing copolymer rubber and the thermoplastic polyurethane.

In addition, regarding the evaluation results of antistatic properties, numerical values corresponding to “X” of the surface resistance value “10^{X}Ω” are shown in Table 1. For example, in the case of Example 1, since the surface resistance value of the test specimen was 10^{6.1}Ω, “6.1” is shown in Table 1.

As clearly seen from Table 1, the molded products (test specimens) of the composite materials obtained in the respective Examples had rubber elasticity and superior gasoline resistance, ozone resistance, and antistatic properties. In addition, all the composite materials were easily prepared.

In addition, when Example 1 is compared to Example 4, the molded product of the composite material obtained in Example 4 in which medium-nitrile NBR having a low bound AN content was used had a lower brittle temperature and superior low-temperature properties.

In addition, when Example 1 is compared to Example 5, the molded product of the composite material obtained in Example 1 in which the amount of the metal salt was high had a lower surface resistance value and superior antistatic properties.

On the other hand, the molded product of the composite material obtained in Comparative Example 1 into which the polyester-based TPU was not incorporated had low ozone resistance, low-temperature properties, and low gasoline resistance.

The molded product of the composite material obtained in Comparative Example 2 into which the nitrile group-containing copolymer rubber was not incorporated had low rubber elasticity.

The molded product of the composite material obtained in Comparative Example 3 in which the polyether-based TPU was used as the thermoplastic polyurethane instead of the polyester-based TPU had low gasoline resistance. In addition, when the test specimen was examined after the evaluation of gasoline resistance, the molded product of the composite material obtained in Comparative Example 3 was partially dissolved.

The molded product of the composite material obtained in Comparative Example 4 in which the amount of the metal salt was lower than 0.1 parts by mass had a high surface resistance value and low antistatic properties.

The composite materials obtained in Comparative Examples 5 and 7 in which the amount of the metal salt was high at 10.0 parts by mass or 5.0 parts by mass had an excess amount of polyalkylene oxide components and thus were in the oil form and could not be molded.

The molded product of the composite material obtained in Comparative Example 6 in which the acrylic resin was used instead of the polyester-based TPU had significantly low ozone resistance. In addition, the molded product of the composite material obtained in Comparative Example 6 had a high surface resistance value and low antistatic properties. The reason is presumed to be as follows. Since the acrylic resin which was used instead of the polyester-based TPU had a carbon skeleton, electron transfer was difficult to occur therein, and insulation properties were superior. Therefore, the surface resistance value of the molded product was high.

What is claimed is:

1. A composite material comprising:
nitrile group-containing copolymer rubber;
polyester-based thermoplastic polyurethane; and
polyalkylene oxide containing metal salt,
wherein the metal salt is alkali metal salt or alkaline earth metal salt, and
an amount of the metal salt in the polyalkylene oxide containing metal salt is 0.5 parts by mass to 3.0 parts by mass with respect to 100 parts by mass of a total amount of the nitrile group-containing copolymer rubber and the polyester-based thermoplastic polyurethane.

2. The composite material according to claim 1,
wherein the polyester-based thermoplastic polyurethane is crosslinked with the polyalkylene oxide containing metal salt.

3. An industrial endoscope comprising:
a member obtained by molding the composite material according to claim 1.

4. The industrial endoscope according to claim 3,
wherein the member is a tube which covers an outer surface of an insertion portion used to be inserted into a tissue.
